(19) United States
(12) Patent Application Publication
(10) Pub. No.: US 2022/0305059 A1
(43) Pub. Date: Sep. 29, 2022

(54) TREATMENT OF AUTISM SPECTRUM DISORDER AND ASSOCIATED NEUROINFLAMMATION USING FIBROBLASTS AND DERIVATIVES THEREOF

(71) Applicant: Figene, LLC, Houston, TX (US)

(72) Inventors: Thomas Ichim, San Diego, CA (US); Pete O'Heeron, Houston, TX (US)

(21) Appl. No.: 17/596,889

(22) PCT Filed: Jun. 22, 2020
(86) PCT No.: PCT/US2020/038967

§ 371 Date: Dec. 20, 2021

Related U.S. Application Data
(60) Provisional application No. 62/864,503, filed on Jun. 21, 2019.

(51) Int. Cl. A61K 35/33; A61P 25/00

(52) U.S. Cl. CPC A61K 35/33; A61P 25/00; A61K 2035/122

(57) ABSTRACT

Disclosed are means, methods, and compositions of matter useful for treatment of pervasive developmental disorders. The treatment includes the use of fibroblasts, modified fibroblasts, and derivatives thereof for reduction of neuroinflammation and/or gastrointestinal inflammation in a patient in need of treatment, such as having a pervasive developmental disorder. Fibroblasts, modified fibroblasts, and derivatives thereof may be administered at a frequency and concentration sufficient to reduce interleukin-17 production in the gut of patients with autism spectrum disorder.

This application claims priority to U.S. Provisional Patent Application Ser. No. 62/864,503, filed Jun. 21, 2019, which is incorporated by reference herein in its entirety.

### TECHNICAL FIELD

Embodiments of the disclosure include at least the fields of molecular biology, cell biology, neurobiology, and medicine.

### BACKGROUND

Autism has reached epidemic proportions with a reported incidence of approximately 1 in 150 newborns [1]. Despite improvements in our understanding of this condition, no curative therapeutic interventions exist. Clinical trials being conducted at present are focused primarily on typical and atypical antipsychotics, as well as various pharmacological approaches targeting symptomology [2]. Many of the medical approaches have drawn fire from parents and patient activist groups for making their children into “walking zombies on all those drugs” [3]. Accordingly, novel therapeutic directions are desperately needed for approaching the problem of autism.

Numerous anatomical and physiological alterations have been reported in patients with autism spectrum disorder (ASD), however their significance is a matter of debate. Redcay and Courchesne overviewed 15 studies using head circumference and MRI assessments to quantify brain growth during development. They found brain size in children with autism was slightly reduced at birth, dramatically increased within the first year of life, but then plateaued so that by adulthood the majority of patients remained within normal range [4]. In another study, a one year longitudinal MRI examination of 70 participants (45 with ASD and 25 controls) demonstrated that the amygdala of children with ASD grew at accelerated rate during ages of 2 to 4 [5]. Older children also appear to have developmental abnormalities. For example a study in patients of an average age of 12-years old examined MRI scans of 13 autistic and 7 non-autistic controls. It was found that autistic boys showed abnormally slowed white matter development, which was most pronounced in the parietal, temporal, and occipital lobes as well as abnormal overgrowth in gray matter structures such as the putamen and anterior cingulate cortex [6]. Other abnormalities that have been associated with autism include attenuated growth of neuron fibers [7], enhanced oxidative stress [8-10], high secretion of amyloid protein breakdown products [11], and impaired ability to utilize the subcortical brain regions involved in face detection and automatic emotional face processing [12]. These diverse observations have been difficult to synthesize into a common underlying biological theme.

Abnormal immune activity has been implicated in the neurodevelopment disorder of ASD. A recent study of a cohort of children with ASD demonstrated and ASD-associated systemic and intestinal immune dysregulation similar to that found in Crohn's disease (CD). CD is a chronic inflammatory disorder of the gastrointestinal tract driven by activated type 1 helper T-cells which results from a deregulated mucosal immune response to normal constituents of the gut microflora (3a).

Initial suggestions supporting an association between inflammation and ASD were made by observations that GI symptoms such as abdominal pain, as well as diarrhea and constipation, are significantly more prevalent in patients with ASD as compared to age-matched controls. For example, one study found that between 9 to 70% of ASD patients had one or more GI manifestations [13]. The wide variation of prevalence in the study was ascribed to differing definitions of ASD and of GI symptoms. The specific symptomatology of ASD GI manifestations has been reported to be similar in nature to inflammatory bowel disease. For example Horvath and Perman [14], noted not only presence of inflammation in both the upper and lower intestinal tract, but also decreased sulfation capacity of the liver, pathologic intestinal permeability, increased secretory response to intravenous secretin injection, and decreased digestive enzyme activity. The authors observed that these manifestations seemed to correlate with regressive behavior. It is important to note that although the existence of GI abnormalities is fairly established, their manifestations are highly heterogenous. For example, Molloy et al examined 137 autistic patients and found that only 24% had a history of at least one chronic gastrointestinal symptom and that the most common symptom was diarrhea, which occurred in 17 percent [15]. However, there are some suggestions that the subset of patients experiencing GI pathology is actually afflicted with a more severe form of ASD. For example, Nikolov et al [16], studied 172 autistic patients of which 39 (22.7%) exhibited GI symptoms, primarily constipation and diarrhea. Of the patients expressing GI manifestations, a greater symptom severity on measures of irritability, anxiety, and social withdrawal was observed.

Although controversial, mucosal lesions in the form of chronic ileocolonic lymphoid nodular hyperplasia characterized by lymphocyte infiltration, complement deposition, and cytokine production have been described specifically in autistic children but not in healthy controls or in cerebral palsy patients [17, 18]. A possible manifestation of this localized inflammation is the “leaky gut” syndrome that has been demonstrated by numerous groups in autistic patients [19], as well as first-degree relatives of patients with ASD[20]. One recent study examined 58 patients with ASD and 39 age-matched controls. It was found that autistic patients had much lower levels of total short chain fatty acids, as well as abnormalities in their bacterial flora with lower number of Bifidobacter species and higher levels of species of Lactobacillus. Furthermore, this study also confirmed that increased gastrointestinal inflammation positively correlated with severity of autistic symptoms [21]. Interestingly, there is some evidence that leaky gut may actually be not only a result of inflammation, but also a propagation factor expressing through systemic release of endotoxins [22]. While clinical evidence is lacking, the notion that probiotics may alter the gut milieu, thus decreasing localized inflammation, has been accepted by numerous clinicians in that a recent US survey has reported that up to one-fifth of complementary medicine physicians encourage the use of probiotics for children with ASD [23]. As such, there is a need in the art to address an inflammatory basis for ASD.

### BRIEF SUMMARY

The present disclosure is directed to a system, method, and compositions for the treatment or prevention of one or more pervasive developmental disorders in an individual. The pervasive developmental disorders treated by embodiments of the disclosure may include Rett syndrome and/or autism spectrum disorders, such as autism, Asperger's, childhood disintegrative disorder, or pervasive developmental disorder not otherwise specified (PDD-NOS), as examples. The individual may be administered one or more compositions that alleviate one or more causes and/or one or more symptoms of a pervasive developmental disorder. In some embodiments, the one or more compositions administered to an individual may inhibit or reduce TNF-alpha and/or interleukin (IL)-17 in the individual. In certain embodiments, the composition(s) administered to an individual stimulate angiogenesis (such as by differentiating into cells of the vasculature and/or providing trophic support) and/or may stimulate neurogenesis (including in the dentate gyrus and/or subventrical zone).

The composition of the disclosure may comprise cells, derivatives of cells, apoptotic bodies of cells, fragments of cells, or a combination thereof. The composition may comprise fibroblasts, derivatives of fibroblasts, apoptotic bodies of fibroblasts, or a combination thereof. Cells of the present disclosure, including fibroblasts, may express CXCR4. Cells of the present disclosure may have an expression of CXCR4 higher than the expression of CXCR4 in mesenchymal stem cells of a similar origin, as one example. In some embodiments, apoptotic bodies may be generated by exposing cells, such as fibroblasts, to one or more DNA damaging agents, such as ultraviolet light and/or a sensitizing agent (increases DNA damage to cells after exposure to UV). For example, sensitizing agents may be considered compounds that are capable of augmenting the DNA damaging and/or cell death inducing abilities of radiation. In one embodiment, UV irradiation is utilized together with 8-psoralen, wherein said 8-psoralen is the sensitizing agent. Psoralen (also called psoralene) is the parent compound in a family of naturally occurring organic compounds known as the linear furanocoumarins. It is structurally related to coumarin by the addition of a fused furan ring, and may be considered as a derivative of umbelliferone. Psoralen occurs naturally in the seeds of Psoralea corylifolia, as well as in the common fig, celery, parsley, West Indian satinwood and in all citrus fruits. It is widely used in PUVA (psoralen +UVA) treatment for psoriasis, eczema, vitiligo, and cutaneous T-cell lymphoma.

Cells of the present disclosure may be from any source, including bone marrow, placental matrix, adipose tissue, menstrual blood, endometrium, muscle, circulating blood, cord blood, or a combination thereof. The cells may be from an autologous source or allogenic source with respect to an individual of the present disclosure. Derivatives and/or apoptotic bodies of cells, including derivatives and/or apoptotic bodies of fibroblasts, may be syngeneic to other cells of the present disclosure.

In some embodiments, compositions of the present disclosure comprise a kit.

The composition administered to an individual of the present disclosure may comprise cell conditioned media, including fibroblast-conditioned media.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWING

For a more complete understanding of the present disclosure, reference is now made to the following descriptions taken in conjunction with the accompanying drawing.

FIG. 1 shows tested models of autism spectrum disorder exposed to control saline, valproic acid (VPA), or VPA followed by fibroblasts (in the groupings of bar graphs, from left to right).

### DETAILED DESCRIPTION

### Definitions

“Allogeneic,” as used herein, refers to cells of the same species that differ genetically from cells of a host.

“Autologous,” as used herein, refers to cells derived from the same subject. The term “engraft” as used herein refers to the process of stem cell incorporation into a tissue of interest in vivo through contact with existing cells of the tissue.

As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the terms “carrier” and “diluent” refers to a pharmaceutically acceptable (e.g., safe and non-toxic for administration to a human) carrier or diluting substance useful for the preparation of a pharmaceutical formulation. Exemplary diluents include sterile water, bacteriostatic water for injection (BWFI), a pH buffered solution (e.g. phosphate-buffered saline), sterile saline solution, Ringer's solution or dextrose solution.

As used herein, the terms “dosage form” and “unit dosage form” refer to a physically discrete unit of a therapeutic agent for the individual to be treated. Each unit contains a predetermined quantity of active material calculated to produce the desired therapeutic effect. It will be understood, however, that the total dosage of the composition will be decided by the attending physician within the scope of sound medical judgment.

A “dosing regimen” (or “therapeutic regimen”), as used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, the therapeutic agent is administered continuously over a predetermined period. In some embodiments, the therapeutic agent is administered once a day (QD) or twice a day (BID), or more.

The term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being administered.

The term “innate immune response” will be understood by the skilled person to be effected by cells and mechanisms that defend the host from infection by other organisms in a non-specific manner, i.e., the cells of the innate system recognize and respond to pathogens in a generic way, but unlike the adaptive immune system, it does not confer long-lasting or protective immunity to the host. Cells of the innate immune response include phagocytes, such as macrophages, neutrophils, dendritic cells, basophils and eosinophils, natural killer cells and γδT cells. The complement system also forms a component of the innate immune system. An innate immune response can induce an adaptive immune response. In some embodiments of the disclosure, administration of fibroblasts, and/or derivatives of fibroblasts, may be used to reduce chronic inflammation and stimulate iron utilization so as to decrease anemia. Autism is associated with higher innate immune response, and fibroblasts decrease this in the present disclosure.

Cells, Modified Cells, Derivatives of Cells, Apoptotic Bodies, and Conditioned Media

Certain aspects of the disclosure concern the use of cells (including fibroblasts), modified cells (including modified fibroblasts), derivatives of cells (including derivatives of fibroblasts), apoptotic bodies from cells (including apoptotic bodies from fibroblasts), and/or conditioned media from cells (including from fibroblasts) for treatment of pervasive developmental disorders, including inflammation associated with said disorders. The cells, modified cells, derivatives of cells, apoptotic bodies from cells, and/or conditioned media may comprise or produce one or more anti-inflammatory factors. The cells, modified cells, derivatives of cells, apoptotic bodies from cells, and/or conditioned media may suppress, reduce, or inhibit IL-17 and/or TNF-alpha, including TNF-alpha produced by activated macrophages. Fibroblasts and/or modified fibroblasts may express CXCR4, including expressing CXCR4 at a higher level than mesenchymal stem cells of similar origin.

Fibroblasts utilized in methods and disclosures encompassed herein may be from any source including, but not limited to, bone marrow, placental matrix, adipose tissue, menstrual blood, endometrium, muscle, circulating blood, cord blood, and a combination thereof.

In some embodiments, fibroblasts of any kind, including placental fibroblast cells, are identified for certain embodiments of the present disclosure based on at least the expression of one or more antigens including Oct-4, Rex-1, CD9, CD13, CD29, CD44, CD166, CD90, CD105, SH-3, SH-4, TRA-1-60, TRA-1-81, SSEA-4, Sox-2, or a combination thereof. Fibroblasts identified or otherwise known to express the one or more antigens may be utilized in methods and compositions of the disclosure.

In certain embodiments, bone marrow fibroblasts may be isolated from bone marrow and utilized in methods and compositions related to treatment or prevention of one or more pervasive developmental disorders. The bone marrow fibroblast cells may be generated from bone marrow derived mononuclear cells, said mononuclear cells containing populations capable of differentiating into one or more of the following cell types: endothelial cells, smooth muscle cells, and neuronal cells. In some embodiments, bone marrow fibroblast cells may be selected based on expression of one or more of the following antigens: CD34, c-kit, flk-1, Stro-1, CD105, CD73, CD31, CD56, CD146, vascular endothelial-cadherin, CD133 and CXCR-4.

In certain embodiments of the disclosure, fibroblasts cells are collected from amniotic fluid or amniotic membrane. The amniotic derived fibroblast cells may be utilized therapeutically in an unpurified manner subsequent to matching. The amniotic fibroblast cells are administered locally, intramuscularly or systemically in a patient suffering from autism. In other embodiments, amniotic fibroblast cells are substantially purified based on expression of markers such as SSEA-3, SSEA4, Tra-1-60, Tra-1-81 and/or Tra-2-54, and subsequently administered. In some embodiments, cells are cultured, as described in U.S. patent application Ser. No. 10/918,739 to Haas, the disclosure of which is incorporated herein by reference, expanded, and subsequently administered (for example, by infusion) into the individual. Amniotic fibroblast cells are described in the following references [31-33]. One particular aspect of amniotic fibroblast cells that may make them amenable for use in practicing certain aspects of the current disclosure is their bi-phenotypic profile as being both mesenchymal and endothelial progenitors this allows for anti-inflammatory, as well as angiogenic function [32, 34]. This property is useful for treatment of individuals with autism that would benefit from angiogenesis, but also from the anti-inflammatory effects of fibroblast cells. The use of amniotic fluid fibroblast cells is particularly useful in situations such as ischemia-associated pathologies and/or inflammatory states, in which hypoxia is known to perpetuate degenerative processes.

In some embodiments, fibroblast cells are isolated from a sample or biopsy of bodily tissue upon enzymatic digestion, mechanical separation, filtration, centrifugation, or a combination thereof. The number and quality of the isolated fibroblast cells can vary depending, for example, on the quality of the tissue used, the compositions of perfusion buffer solutions, and/or the type and concentration of enzyme. Frequently used enzymes include, but are not limited to, collagenase, pronase, trypsin, dispase, hyaluronidase, thermolysin and pancreatin, and combinations thereof. Collagenase is most commonly used, often prepared from bacteria (e.g., from Clostridium histolyticum), and may often consist of a poorly purified blend of enzymes, which may have inconsistent enzymatic action. Some of the enzymes exhibit protease activity, which may cause unwanted reactions affecting the quality and quantity of viable/healthy fibroblast cells. It is understood by those of skill in the art to use enzymes of sufficient purity and quality to obtain viable fibroblast cell populations.

Certain methods of the disclosure concern culturing fibroblast cells obtained from human tissue samples. In some embodiments, the populations of fibroblast cells are plated onto a substrate. In the present disclosure, fibroblasts may be plated onto a substrate which allows for adherence of cells thereto. This may be carried out, for example, by plating the cells in a culture plate which displays one or more substrate surfaces compatible with cell adhesion. When the said one or more substrate surfaces contact the suspension of cells (e.g., suspension in a medium) introduced into the culture system, cell adhesion between the cells and the substrate surfaces may ensue. Accordingly, in certain embodiments cells are introduced into a culture system which features at least one substrate surface that is generally compatible with adherence of cells thereto, such that the plated cells can contact the said substrate surface, such embodiments encompass plating onto a substrate, which allows adherence of cells thereto. General principles of maintaining adherent cell cultures are well-known in the art. As appreciated by those skilled in the art, the fibroblast cells may be counted in order to facilitate subsequent plating of the cells at a desired density. Where, as in the present disclosure, the cells after plating may primarily adhere to a substrate surface present in the culture system (e.g., in a culture vessel), the plating density may be expressed as number of cells plated per mm² or cm² of the said substrate surface. In practicing the disclosure, after plating of the fibroblasts, the cell suspension is left in contact with the adherent surface to allow for adherence of cells from the cell population to the said substrate. In contacting fibroblasts to the adherent substrate, the cells may be advantageously suspended in an environment comprising at least a medium, in the methods of the disclosure typically a liquid medium, which supports the survival and/or growth of the cells. The medium may be added to the system before, together with or after the introduction of the cells thereto. The medium may be fresh, i.e., not previously used for culturing of cells, or may comprise at least a portion which has been conditioned by prior culturing of cells therein, e.g., culturing of the cells which are being plated or antecedents thereof, or culturing of cells more distantly related to or unrelated to the cells being plated.

In some embodiments, cells, including fibroblasts, are modified to enhance properties useful for aspects of the disclosure. Modifications to cells include, but are not limited to, exposure to external signals, hypoxia, growth factors, dedifferentiating agents, differentiating agents, conditioned media, or a combination thereof. Cells may be modified to express proteins and/or nucleic acids useful for the present invention. Expression of proteins and/or nucleic acids may be generated by any method known in the art.

Some embodiments of the present disclosure concern the use of derivatives of cells, or products made from cells, including derivatives of fibroblasts. Some embodiments of the present disclosure concern the use of apoptotic bodies as derivatives of fibroblasts, including apoptotic bodies from fibroblasts of any kind. Apoptotic bodies may be capable of immune regulation, including regulating inflammation. Apoptotic bodies may release molecules that signal to the immune system, which may activate immune mechanisms. Apoptotic bodies may be generated by any method known in the art, such as by exposing cells to one or more DNA damaging agents, such as ultraviolet light and/or a sensitizing agent.

Certain embodiments concern the use of cell-conditioned media, including fibroblast-conditioned media. Conditioned media may contain molecules useful for embodiments of the present disclosure, including molecules that regulate inflammation, such as IL-17.

In some embodiments, cells (including fibroblasts), modified cells (including modified fibroblasts), derivatives of cells (including derivatives of fibroblasts), apoptotic bodies from cells (including apoptotic bodies from fibroblasts), and/or conditioned media from cells (including from fibroblasts) are combined. In specific cases, the combination may include the combination of syngeneic cells (including fibroblasts), syngeneic modified cells (including modified fibroblasts), derivatives of syngeneic cells (including derivatives of fibroblasts), apoptotic bodies from syngeneic cells (including apoptotic bodies from fibroblasts), and/or conditioned media from syngeneic cells (including from fibroblasts).

In some embodiments, the cells of the present disclosure may be cultured for at least between about 10 days and about 40 days, for at least between about 15 days and about 35 days, for at least between about 15 days and 21 days, such as for at least about 15, 16, 17, 18, 19 or 21 days. In some embodiments, the cells of the disclosure may be cultured for no longer than 60 days, or no longer than 50 days, or no longer than 45 days. In some embodiments, fibroblasts (including modified fibroblasts) are cultured in the presence of a liquid culture medium. Typically, the medium will comprise a basal medium formulation as known in the art. Many basal media formulations can be used to culture fibroblasts herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, and modifications and/or combinations thereof. Compositions of the above basal media are generally known in the art and it is within the skill of one in the art to modify or modulate concentrations of media and/or media supplements as necessary for the fibroblasts cultured. In some embodiments, a culture medium formulation may be explants medium (CEM) which is composed of IMDM supplemented with 10% fetal bovine serum (FBS, Lonza), 100 U/ml penicillin G, 100 μg/ml streptomycin and 2 mmol/L L-glutamine (Sigma-Aldrich). Other embodiments may employ further basal media formulations, such as chosen from the ones above.

For use in the fibroblast culture, including modified fibroblasts culture, media can be supplied with one or more further components. For example, additional supplements can be used to supply the cells with the necessary trace elements and substances for optimal growth and expansion. Such supplements include insulin, transferrin, selenium salts, and combinations thereof. These components can be included in a salt solution such as, but not limited to, Hanks' Balanced Salt Solution (HBSS), Earle's Salt Solution. Further antioxidant supplements may be added, e.g., β-mercaptoethanol. While many media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin. Also contemplated is supplementation of cell culture medium with mammalian plasma or sera. Plasma or sera often contain cellular factors and components that are necessary for viability and expansion. The use of suitable serum replacements is also contemplated (e.g., FBS). In some embodiments, culturing tissue explants and fibroblast cells for time durations as defined herein, and preferably using media compositions as described herein results in the emergence and proliferation of a progenitor or stem cell of the disclosure. In some embodiments, fibroblast cells of the present disclosure are identified and characterized by their expression of specific marker proteins, such as cell-surface markers. Detection and isolation of these cells can be achieved, e.g., through flow cytometry, ELISA, and/or magnetic beads. Reverse-transcription polymerase chain reaction (RT-PCR) can also be used to monitor changes in gene expression in response to differentiation. Methods for characterizing fibroblasts the present disclosure are provided herein.

In some embodiments, the fibroblast are cultured in a manner to increase the activities or capabilities useful for the methods disclosed herein. The fibroblasts may be cultured to promote the ability of the fibroblasts to reduce inflammatory mediator production and/or to promote their ability to have regenerative activity. In some embodiments, the fibroblasts, including fibroblasts able to reduce inflammatory mediator production, are cultured in the presence of tissue culture additives, such as interleukin-10, indomethacin, valproic acid, low dose naltrexone, interleukin-27, or a combination thereof, for example. In specific embodiments of the disclosure, regenerative cells such as fibroblasts are utilized to treat autism spectrum disorder and the cells may be exposed to one of the aforementioned additives in culture, such as valproic acid, to increase their potency.

In some embodiments, any combination of the cells (including fibroblasts), modified cells (including modified fibroblasts), derivatives of cells (including derivatives of fibroblasts), apoptotic bodies from cells (including apoptotic bodies from fibroblasts), and/or conditioned media from cells (including from fibroblasts) are comprised in a kit.

Pervasive Developmental Disorders

Embodiments of the present disclosure concern the treatment or prevention of one or more pervasive developmental disorders. An individual having, or diagnosed with having, a pervasive developmental disorder may have social and/or communication developmental delays, for example. Pervasive developmental disorders may include any autism spectrum disorder, such as autism, Asperger's, childhood disintegrative disorder, or pervasive developmental disorder not otherwise specified (PDD-NOS). The pervasive developmental disorder may be Rett syndrome, in at least some cases.

Pervasive developmental disorders may present with or be caused by abnormal molecular markers, including abnormal inflammation, in an individual. Such markers may include abnormal levels of CRP, erythrocyte sedimentation ratio, fibrinogen, interleukin-1, TNF-alpha, prohepcidin, hepcidin, interleukin-6, interleukin-17, interleukin-18, interleukin-12, interleukin-18, interleukin-23, interleukin-27, interleukin-33, interferon-gamma, HMBG-1, calreticulin. Other inflammatory markers useful for assessment of conditions associated with anemia of chronic disease with the practice of the disclosure include Apo A1 (Apolipoprotein A1), Beta-2 Microglobulin, Clusterin, CRP (C Reactive Protein), Cystatin-C, Eotaxin, Factor VII, FGF-9 (Fibroblast Growth Factor-9), GCP-2 (Granulocyte Chemotactic Protein-2), Growth Hormone, IgA (Immunoglobulin A), IL-10 (Interleukin-10), IL-1beta (Interleukin-1beta), IL-2 (Interleukin-2), IL-4 (Interleukin-4), IL-5 (Interleukin-5), Insulin, IP-10 (Inducible Protein-10), Leptin, LIF (Leukemia Inhibitory Factor), MDC (Macrophage-Derived Chemokine), MIP-1 alpha (Macrophage Inflammatory Protein-1alpha), MIP-1beta (Macrophage Inflammatory Protein-1beta), MIP-1gamma (Macrophage Inflammatory Protein-1gamma), MIP-2 (Macrophage Inflammatory Protein-2), MIP-3beta (Macrophage Inflammatory Protein-3beta), MPO (Myeloperoxidase), Myoglobin, NGAL (Lipocalin-2), OSM (Oncostatin M), Osteopontin, SAP (Serum Amyloid P), SCF (Stem Cell Factor), SGOT (Serum Glutamic-Oxaloacetic Transaminase), TIMP-1 (Tissue Inhibitor of Metalloproteinase Type-1), Tissue Factor, TPO (Thrombopoietin), VEGF (Vascular Endothelial Cell Growth Factor), or a combination thereof.

Individuals with pervasive developmental disorders may present with neuro-inflammation and/or gastrointestinal inflammation. The personal developmental disorder may be caused, fully or in part, by neuro-inflammation and/or gastrointestinal inflammation, in certain embodiments.

In some cases, the methods and compositions of the disclosure concern prevention of one or more pervasive developmental disorders. In specific cases, the prevention concerns the complete absence of the disorder or the delay in onset and/or severity of the order. An individual may be provided effective amounts of the compositions of the disorder in a situation in which the individual is from a family with history and/or when an individual has been identified as having or susceptible to having one or more pervasive developmental disorders.

Administration

Certain aspects of the disclosure concern administration of compositions, which may include fibroblasts, modified fibroblasts, derivatives of at least one fibroblast, fibroblast apoptotic bodies, fibroblast-conditioned media, or a combination thereof, for the purpose of treating pervasive developmental disorders. Treating pervasive developmental disorders may (or in some cases, may not) include decreasing inflammation associated with pervasive developmental disorders, such as autism.

In some embodiments, an individual suffering from a pervasive developmental disorder, such as autism, may be selected for treatment disclosed herein based on inflammatory status. Said status may be identified by abnormally high serum levels of CRP, erythrocyte sedimentation ratio, fibrinogen, interleukin-1, and/or TNF-alpha. In some embodiments, inflammatory status of an individual is identified by abnormally high ability of monocytes derived from the individual to produce TNF-alpha upon stimulation of toll like receptor 4 using agents such as lipopolysaccharide. Administration of compositions of the present disclosure may reduce inflammatory status markers as disclosed herein. In alternative cases, an individual is provided effective amounts of methods and compositions of the disclosure regardless of whether there are high serum levels of the factors listed above.

In some embodiments, individuals that are eligible to receive the therapy possess higher levels of inflammatory markers as compared to age-matched controls Inflammatory markers include C-reactive protein (CRP), fibrinogen, and the erythrocyte sedimentation rate (ESR), prohepcidin, hepcidin, TNF-alpha, interleukin-1, interleukin-6, interleukin-17, interleukin-18, interleukin-12, interleukin-18, interleukin-23, interleukin-27, interleukin-33, interferon-gamma, HMBG-1, calreticulin. Other inflammatory markers useful for assessment of conditions associated with anemia of chronic disease with the practice of the disclosure include Apo A1 (Apolipoprotein A1), Beta-2 Microglobulin, Clusterin, CRP (C Reactive Protein), Cystatin-C, Eotaxin, Factor VII, FGF-9 (Fibroblast Growth Factor-9), GCP-2 (Granulocyte Chemotactic Protein-2), Growth Hormone, IgA (Immunoglobulin A), IL-10 (Interleukin-10), IL-1beta (Interleukin-1beta), IL-2 (Interleukin-2), IL-4 (Interleukin-4), IL-5 (Interleukin-5), Insulin, IP-10 (Inducible Protein-10), Leptin, LIF (Leukemia Inhibitory Factor), MDC (Macrophage-Derived Chemokine), MIP-1alpha (Macrophage Inflammatory Protein-1alpha), MIP-1beta (Macrophage Inflammatory Protein-1beta), MIP-1gamma (Macrophage Inflammatory Protein-1gamma), MIP-2 (Macrophage Inflammatory Protein-2), MIP-3beta (Macrophage Inflammatory Protein-3beta), MPO (Myeloperoxidase), Myoglobin, NGAL (Lipocalin-2), OSM (Oncostatin M), Osteopontin, SAP (Serum Amyloid P), SCF (Stem Cell Factor), SGOT (Serum Glutamic-Oxaloacetic Transaminase), TIMP-1 (Tissue Inhibitor of Metalloproteinase Type-1), Tissue Factor, TPO (Thrombopoietin), VEGF (Vascular Endothelial Cell Growth Factor), or a combination thereof. Administration of compositions of the present disclosure may reduce inflammatory markers as disclosed herein. In alternative cases, an individual is provided effective amounts of methods and compositions of the disclosure regardless of whether there are high levels of the factors listed above.

Individuals identified as suitable for treatment may be administered fibroblast products based on degree of underlying inflammation. In some embodiments, an individual may be administered intravenously a population of non-immunogenic fibroblasts at a concentration sufficient to reduce and/or ameliorate inflammation and/or neuroinflammation. In some embodiments, conditioned media of fibroblasts is administered. In some embodiments, apoptotic bodies of fibroblasts are administered.

In certain aspects of the disclosure, one or more anti-inflammatory agents may be administered to an individual receiving cells and/or cell derivatives of the present disclosure that increase perfusion and stimulate neurogenesis, said inflammatory inhibiting agents may inhibit molecular pathways such as the NF-kappa B pathway, the MyD88 pathway, the TNF signal transduction pathway, the Toll like receptor signal transduction pathway, and other pathways associated with upregulation of MHC expression, upregulation of C-reactive protein production, and upregulation of TNF alpha production. Anti-inflammatory agents useful for practice of the disclosure are well known in the art and include Alclofenac; Alclometasone Dipropionate; Algestone Acetonide; Alpha Amylase; Alpha-lipoic acid; Alpha tocopherol; Amcinafal; Amcinafide; Amfenac Sodium; Amiprilose Hydrochloride; Anakinra; Anirolac; Anitrazafen; Apazone; Ascorbic Acid; Balsalazide Disodium; Bendazac; Benoxaprofen; Benzydamine Hydrochloride; Bromelains; Broperamole; Budesonide; Carprofen; Chlorogenic acid; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Ellagic acid; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Glutathione; Halcinonide; Halobetasol Propionate; Halopredone Acetate; Hesperedin; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Lycopene; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Oleuropein; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Pycnogenol; Polyphenols; Prednazate; Prifelone; Prodolic Acid; Proquazone; Proxazole; Proxazole Citrate; Quercetin; Reseveratrol; Rimexolone; Romazarit; Rosmarinic acid; Rutin; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrahydrocurcumin; Tetrydamine; Tiopinac; Tixocortol Pivalate; Tolmetin; Tolmetin Sodium; Triclonide; Triflumidate; Zidometacin; Zomepirac Sodium, IL-4, IL-10, IL-13, IL-20, IL-1 receptor antagonist, and TGF-beta.

In some embodiments, fibroblast cells, such as bone marrow fibroblast cells, are administered as described herein in combination with agents known to increase regenerative activity. Such agents may include, for example, erythropoietin [24], prolactin [25], human chorionic gonadotropin (as described in U.S. Pat. No. 5,968,513 and incorporated by reference), gastrin [26], EGF [27], FGF [28], and/or VEGF [29]. In certain embodiments, administration of neutralizers of TNF alpha are concurrently administered with fibroblasts to derepress inhibitory effects of this cytokine on circulating fibroblasts cells [30].

In some embodiments, administration of compositions of the present disclosure stimulate angiogenesis and/or neurogenesis. In some embodiments, angiogenesis is stimulated by cells of the presented disclosure differentiating into cells of the vasculature. Angiogenesis may be stimulated by providing trophic support to the cells of vasculature. Neurogenesis may be stimulated anywhere in an individual including, in the dentate and subventrical zone.

In certain embodiments, administration of cell-conditioned media, including fibroblast-conditioned media may be performed at a concentration and frequency sufficient to stimulate neurogenesis. Neurogenesis may be stimulated anywhere in an individual including, in the dentate and subventrical zone. Administration of cell-conditioned media, including fibroblast-conditioned media may suppress interleukin-17 production.

In certain embodiments, administration of compositions of the present disclosure may reduce neuro-inflammation and/or gastrointestinal inflammation.

The compositions, which may include fibroblasts, modified fibroblasts, derivatives of at least one fibroblast, fibroblast apoptotic bodies, fibroblast-conditioned media, or a combination thereof, disclosed herein may be administered to an individual, including an animal, such as a human, by a number of methods known in the art. Examples of suitable methods include intramuscular, intradermal, intraepidermal, intravenous, intraarterial, subcutaneous, or intraperitoneal administration, oral administration, and/or topical application (such as ocular, intranasal, and intravaginal application).

In some embodiments, instructions for administration of compositions, as disclosed herein, are provided.

### EXAMPLES

The following example is included to demonstrate particular embodiments of the disclosure. It should be appreciated by those of skill in the art that the techniques disclosed in the examples that follow represent techniques discovered by the inventors to function well in the practice of the disclosed material, and thus can be considered to constitute particular modes for its practice. However, those of skill in the art should, in light of the present disclosure, appreciate that many changes can be made in the specific embodiments that are disclosed and still obtain a like or similar result without departing from the spirit and scope of the invention.

### Example 1

### Cell Administration Clinical Trial

The example herein describes a clinical trial for assessing the efficacy, feasibility, and safety of fibroblast administration to autism spectrum disorder patients. Patients are administered fibroblasts and evaluated at four visits. Two subsequent visits are used to further evaluate the patients.

Visit 1: Baseline Assessments and Cell Administration (Time Zero)
Questionnaires
qEEG
Sleep qEEG
Blood draw
Cell administration

Visit 2: Second Cell Administration
Questionnaires
Quantitative EEG
Blood draw
Cell administration

Visit 3: Third Cell Administration
Questionnaires
Quantitative EEG
Blood draw
Cell administration

Visit 4: Fourth Cell Administration (9 Months)
Questionnaires
Quantitative EEG
Blood draw
Cell administration

Visit 5: 12 Month Follow-up
Questionnaires
Quantitative EEG
Sleep EEG
Blood draw

Visit 6: 18 Month Follow-up
Questionnaires
Quantitative EEG
Blood draw

Patient Population

Subjects have a screening visit with baseline evaluations performed within 15 days prior to the first dose of cells and meet all inclusion and exclusion criteria. Results of all baseline evaluations, which assure that all inclusion and exclusion criteria have been satisfied, must be reviewed by the Principal Investigator or his/her designee prior to enrollment of that subject. The subject is informed about all aspects of the study and written informed consent must be obtained from the subject prior to study procedures.

Inclusion:
Ages 6-9
DSM IV diagnosis of autism confirmed by ADOS and/or ADI-R
No anticipated changes in treatment for the study duration (e.g., diet, nutrients)
No additional biomedical treatments started 6 weeks prior to enrollment
No changes in dietary management for 3 months prior to enrollment
Ambulatory or require minimum support walking, per parent
Able to sit still for 5 minutes or longer with a preferred toy item, per parent
Adequate vision and hearing for the purposes of test administration, per parent
Adequate arm-hand-finger coordination (i.e., able to point) for learning and cognitive tasks used in outcome measurement, per parent
Stable and controlled medical disorder
Under the care of a caregiver willing to participate by attending regularly scheduled appointments and completing the necessary measures

Exclusion:
Significant prematurity at birth (less than 32 weeks gestation); or birthweight significantly below normal for gestational age (SGA—small for gestational age).
Mental retardation
Seizure disorder
Auto-immune conditions
GI malabsorption
History of head trauma and other neurological or medical conditions.

The “drug substance” for the clinical trial comprises 100 million CybroCell fibroblasts, which are administered intravenously in a 20 ml solution over the period of 15-30 minutes. Administration is performed in 4 cycles: Cycle 1 occurs at Time Points 0, 1 (3 months), 2 (6 months), and 3 (9 months). Each cycle consists of administration of 100 million cells once per day for 4 consecutive days.

Primary outcome variables are safety and feasibility as assessed by quantification of adverse reactions associated with treatment.

Each adverse event is assessed for its severity, or the intensity of an event experienced by the subject, using the following:
Mild (1): Discomfort noticed, but no disruption to daily activity.
Moderate (2): Discomfort sufficient to reduce or affect normal daily activity.
Severe (3): Inability to work or perform normal daily activity.
Disabling AE (4): hospitalized

Secondary outcome variables are efficacy signals as quantified by:
Childhood Autism Rating Scale
Autism Behavior Checklist
Vineland Adaptive Behavior Scales
Differential Ability Scales
Children's Global Assessment Scale
Social Responsiveness Scale
Social Skills Rating System
Matson's Evaluation of Social Skills with Youngsters
Autism Quotient
Empathy Quotient
Systematizing Quotient—Revised

Statistical analysis is performed using the SPSS 16.0 software. Safety and exploratory efficacy secondary end-points are observed for each patient against the baseline values. A p value <0.05 is considered statistically significant. The Intent-to-Treat (ITT) population includes all subjects who met eligibility criteria, gave consent to participate, and were treated. The Per Protocol (PP) population is defined as the subgroup of the ITT population with documented adherence to the study protocol. These subjects meet all inclusion and exclusion criteria and have had evaluations at the protocol-specified time points.

Patient demographics and preoperative clinical variables are expressed as percentages or means as appropriate, and are assessed using the student paired t-test analysis: endpoints are compared from the preoperative time point to the 3, 6, 9, 12 and 18 month post first injection time points. If the data are not normally distributed, comparable non-parametric methods are employed.

The trial will be stopped for review if patients under Grade 3 or 4 treatment associated adverse event.

### Example 2

### Fibroblasts for Treatment of Autism

The present example concerns fibroblasts for the treatment of autism, as one example of neuroinflammation.

Male Wistar rats were used in the study. The day of birth was counted as postnatal day (pnd) 0. Animals were housed in a 12-h dark/light cycle in a temperature-controlled environment with food and water ad libitum.

Rats were divided into 3 groups of 10. Group A received Saline. Group B received Valproic Acid on days 6-11 (150 mg/kg). It is known that valproic acid in rodents can mimic autism (Dobrovolsky et al., 2019, J. Transl Med., 17: 400. 17: 400). Group C received Valproic Acid on days 6-11 (150 mg/kg) and Fibroblasts on days 6, 9 and 12 (1 million cells per rat, intravenously, fibroblasts were derived from foreskin and expanded in OptiMem media with 10% fetal calf serum).

At day 32, rats were subjected to a “play test” using a Plexiglas box (30 cm×20 cm×20 cm) that was divided into two equally sized compartments by a clear partition with a semicircular hole (7 cm×5 cm), allowing one animal at a time to move between compartments. Prior to any social interaction, experimental subjects were marked on the back with a black marker and isolated for a total of 30 min (20 min alone in a holding cage+10 min habituation to the testing apparatus) in the dimly lit testing room. During the 10-min habituation period, subjects were allowed to explore the testing box. Following habituation, the treated animal and drug-naïve play partner (matched for sex, age, and weight+/−10 g) was introduced, and their social interactions were observed for 10 minutes

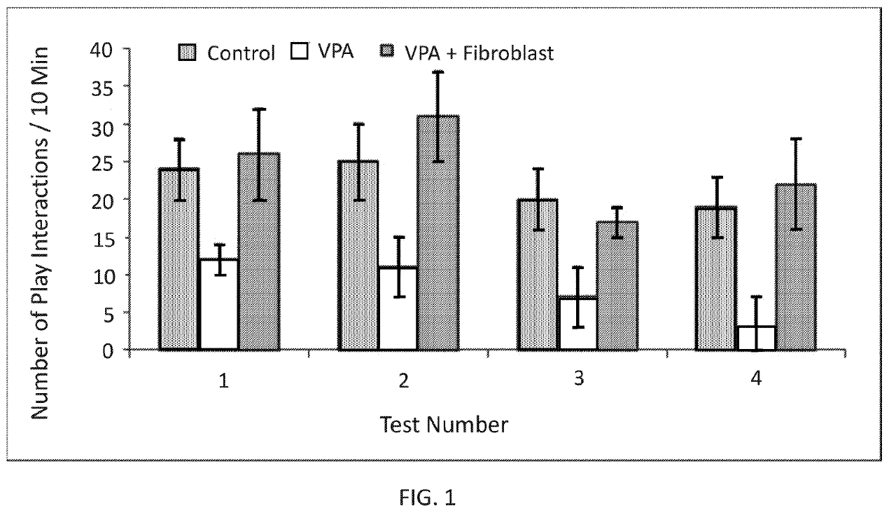

Play fighting (or social play) was defined as pinning, pouncing or playful nape attack, following and chasing. Tests were performed on 4 separate occasions on day 32 (Test 1), day 33 (Test 2), day 34 (Test 3) and day 35 (Test 4). As seen in FIG. 1, a significant decrease in social play was observed in rats treated with valproic acid, which was reversed in rats treated with fibroblasts.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A method of treating a pervasive developmental disorder comprising administering a therapeutically effective amount of a composition comprising fibroblasts, modified fibroblasts, fibroblast apoptotic bodies, fibroblast-conditioned media, or a combination thereof into an individual in need thereof.

2. The method of claim 1, wherein said pervasive developmental disorder is selected from the group consisting of autism or autism spectrum disorder, Rett Syndrome, childhood disintegrative disorder, Asperger's syndrome, pervasive developmental disorder not otherwise specified, and a combination thereof.

3. The method of claim 1, wherein said fibroblasts and/or modified fibroblasts possess expression of CXCR4.

4. The method of claim 1, wherein the composition induces the inhibition of the production of TNF-alpha.

5. The method of claim 1, wherein the composition induces the inhibition of interleukin-17 production.

6. The method of claim 1, wherein the composition stimulates angiogenesis by differentiating into cells of the individual's vasculature or by providing trophic support to cells of the individual's vasculature.

7. The method of claim 1, wherein the fibroblast apoptotic bodies are generated by exposure of fibroblasts to one or more DNA damaging agents.

8. The method of claim 7, wherein at least one DNA damaging agent comprises ultraviolet light, a sensitizing agent, or a combination thereof.

9. The method of claim 1, wherein the administration is at a concentration and frequency sufficient to stimulate neurogenesis in the individual.

10. The method of claim 9, wherein said neurogenesis occurs in the dentate gyrus.

11. The method of claim 9, wherein said neurogenesis occurs in the subventricular zone.

12. The method of claim 1, wherein the fibroblasts, modified fibroblasts, derivatives of at least one fibroblast, fibroblast apoptotic bodies, and/or fibroblast-conditioned media are from a source selected from the group consisting of bone marrow, placental matrix, adipose tissue, menstrual blood, endometrium, muscle, circulating blood, cord blood, and a combination thereof.

13. The method of claim 1, wherein the fibroblasts, modified fibroblasts, derivatives of at least one fibroblast, fibroblast apoptotic bodies, and/or fibroblast-conditioned media are allogenic and/or autologous to the individual.

14. A kit comprising any combination of: fibroblasts, modified fibroblasts, derivatives of at least one fibroblast, fibroblast apoptotic bodies, and fibroblast-conditioned media.

15. The kit of claim 14, wherein the fibroblasts, modified fibroblasts, derivatives of at least one fibroblast, fibroblast apoptotic bodies, and/or fibroblast-conditioned media produce one or more anti-inflammatory factors.

16. The kit of claim 14, wherein the fibroblast apoptotic bodies are syngeneic with said fibroblasts and/or modified fibroblasts.

17. The kit of claim 14, wherein the fibroblasts, modified fibroblasts, derivatives of at least one fibroblast, fibroblast apoptotic bodies, and fibroblast-conditioned media have activity capable of suppressing interleukin-17 production, TNF-alpha production, or both.

18. The kit of claim 14, wherein the fibroblasts, modified fibroblasts, derivatives of at least one fibroblast, fibroblast apoptotic bodies, and fibroblast-conditioned media is collected from a source selected from the group consisting of bone marrow, placental matrix, adipose tissue, menstrual blood, endometrium, muscle, circulating blood, cord blood, and a combination thereof.

19. The kit of claim 14, wherein said fibroblast and/or modified fibroblasts expresses higher concentrations of CXCR4 as compared to a mesenchymal stem cells derived from a similar tissue of origin.

20. The kit of claim 14, wherein the fibroblast apoptotic bodies are generated by exposure of fibroblasts to one or more DNA damaging agents.

21. The kit of claim 20, wherein at least one DNA damaging agent comprises ultraviolet light, a sensitizing agent, or a combination thereof.

22. The kit of claim 14, further comprising instructions for administering said cells treat said pervasive developmental disorder in said individual.
